(19) United States
(12) Patent Application Publication
(10) Pub. No.: US 2018/0275109 A1
(43) Pub. Date: Sep. 27, 2018

(54) Detection of Pesticide Residues in Agriculture

(71) Applicant: Betül Yasemin Akar, Izmir (TR)

(72) Inventor: Betül Yasemin Akar, Izmir (TR)

(21) Appl. No.: 15/749,124

(22) PCT Filed: Jul. 29, 2016
(86) PCT No.: PCT/TR2016/000107

§ 371 Date: Jan. 31, 2018

(30) Foreign Application Priority Data
Jul. 31, 2015 (TR) 2015/09505

(51) Int. Cl. G01N 33/00; G01N 27/49; H01M 10/42

(52) U.S. Cl. CPC G01N 33/0098; G01N 27/49; H01M 2220/30; H01M 2010/4271; H01M 10/4257

(57) ABSTRACT

This invention is related with the portable electronic pesticide analysis device which is used for finding the pesticide or/and the residue of active matter metabolites from pesticide on agricultural products in the pre-harvest area and analyzing methods.

### BACKGROUND OF THE INVENTION

### Field of Invention

This invention is related with the portable electronic pesticide analysis device which is used for finding the pesticide or/and the residue of active matter metabolites from pesticide on agricultural products in the pre-harvest area and analyzing methods.

### Related Art

Because of the harmful effects of pesticide on the human health, there are rules on using pesticide whole around the world and the controls for that can cause serious sanctions. There are given traning to the manufacturers for increasing their awareness on pesticide's harms. Technical experts teach them to how to apply pesticides and the importance of the plant should get rid of it from its system till it harvested. We both teach the manufacturers and also make some residue analysis in periods.

Analysis of pesticide residue in agricultural products are made in laboratories. These labs are each country's official institutions and accredited associations. Products are getting from the production fields according to specified legal sampling method and brought to the lab. In these labs, samples are getting ready for the analysis and then they analyzed with the appropriate device and their report being held. For pesticide residue analysis on agricultural products, the samples must be brought to the laboratory.

In the laboratory, there are highly advanced, comprehensive and sensitive analyzing devices. These devices make the analysis based on chromatographic analysis methods and also these devices are a large and complex-structured device which consists of more than one component system. In the system there is main device which make the analysis, there is monitor which make the register and collect data, there is another computer which makes processing the data, there is a printer and wires which provides energy to the whole machine and it needs an energy source. In addition, all systems are connected with each other.

The samples will be ready for analysis after the process of washing, fragmentation and centrifuge. These chemicals which are used during those process are unhealthy for human and environment.

Analysis of agricultural product's process is long; during the peak season the analysis report can be prepared in couple of days. These could give difficult times for whose dealing with buying and selling of agricultural products and exportation of them.

In some cases, there will be found some over limited pesticide residue on the products which were already loaded for shipment and they had to called the trucks back. In such cases, most of the time exportation is cancelled, but sometimes they export the goods and at the buyer side the residue is found and that caused extermination of the products.

In such cases cause damage for the companies on financial or reputation. They get into those countries black list and that cause damage their future interactions. In addition to that, because of such incidents, countries have gotten negative effects on their prestige. Despite the given training to the manufacturers, there can be seen commercial concerns are more important. According to testimony of the manufacturers, they claimed that there are different party products which is sent to the analysis and to the buyer. Agricultural products are generally transported by larger tonnage trucks which holds many different products from different manufacturer. However according to the regulation, for the analysis the sample should be gotten from different parts of all the loaded products and if there will be found any residue, all the products must be exterminated. In such cases, there cannot be detect the manufacturer who used illegal limit of drugs or no one admits it and the only reliable person will be the trader.

Because of damage of pesticide residue and faced the difficulties during the process, there are many studies going on making the analysis on the field.

In the document of patent no. KR20070004500 A 20070109 there are mentioned hand-held pesticide residue analysis device which can be used in the field. In the device there is a storage bin for water, a water pump for giving the sample water, an electric power supply or battery to rotate the shaft, there is a part which allows you put the rotating part of the motor and contact with the sample, a part that provides measuring the electric current and there is a part which allows you by comparing the value of standard load changes of pesticide analysis. This device in the patent used for analyzing of any multiple systemic pesticide's active substances and active ingredients is mentioned to be on the surface of vegetable and fruit.

There was mentioned an pesticide resudue device in the CN1544916 A 20041110 numbered document. It contains a monochromatic light source, a detector, monochromatic lights are sent and the colorimetric analysis samples in solution tank which keeps the heat in 37 degrees Celsius. This device analysis each sample stabilization for the preset color wavelength light which is sent and where the parts are read with the reaction occurred photoelectric sensor through the converter to the computer on the device being sent and data are processed.

Spectrophotometry, is not one of the electrochemical analysis method. This device is different from the electrochemical methods in pesticide analysis. This device uses Spectrophotometric method in analysis of active pesticide ingredient, for example, genus and specific wavelengths is found to be required only a single example at one time for an item. The device which is mentioned in the patent cannot make pesticide analysis for a group of active pesticide ingredients and multiple active pesticide ingredient metabolites.

In WO 99/United States patent documents numbered 08554 mentioned a portable analysis device appropriate on usage in field. The device contains a microprocessor and a memory card, through the sensors, it can analysis blood, urea, glucose, proteins, pharmaceuticals, pesticides, and water-soluble lead, cadmium, mercury, heavy metal ions. It is used in the device colloidal gold-plated sensors, gold-plated electrodes and electrochemical analysis techniques. The connection between the device and computer can be made with RS232 port through the wires. The most important problem of the RS232 affected by noise on signal line. For this reason, the data transfer can be made between max. 15 meter cables. This affects the analysis sensitivity negatively. In addition, RS232 port is very sensitive for electrical voltage fluctuations. Even with all the protection for such matters, such as avoiding common grounding, any voltage change will effect the results negatively. Considering the land conditions of agricultural areas, it may impossible to provide standard voltage which the device need to make accurate analysis.

The device's software and hardware are working for only running the machine, therefore it needs to install to a computer or/and updated from a computer. The device does not have its own computer operating system.

With this device, there can be made in a variety of analysis in liquids and agricultural drugs. This device mainly used for detecting heavy metals and pesticide residues in the natural water sources. With this device we can make organophosphates and carbamates analyses of pesticides.

In the agricultural drugs, there are not only organophosphates and carbamates pesticides but also different group of pharmaceutical chemicals. Such as chlorinated compounds, synthetic pyrethroid, Amine and hydrazine derivatives, organic Tin, sulfur compounds, triazoles and etc. We cannot claim there is no pesticide residue based on only organophosphates and carbamates pesticides.

The device can make other fluid structure material analysis beside the pesticide such as blood, urine, water is liquid. However, plant's SAP is different from blood and urine in viscosities base. Such as a pepper's SAP and blood serum is very different each other. Before the pesticide analysis, the plant's SAP must be prepared for the machine in the laboratory.

For any laboratory who has the appropriate software and mechanism, these analyses are already performed.

In this device has lack of ability to have such important information such as the location of the land where the sample had, manufacturer information, the date when it is recorded.

This device's purpose is different from the portable electronic pesticide analysis device which is used for finding the pesticide or/and the residue of active matter metabolites from pesticide on agricultural products in the pre-harvest area; in method, principal, condition and working style base.

This device is unable to make analysis on in vivo environment non-liquid plant examples analysis of multiple pesticides.

It is mentioned in developed by a group of researchers MyDx (My Diagnostic) hand-held device made of pesticide residue analysis. The device which is mentioned is a mini olphactometric analysis device. Pesticide active ingredient's essential items in the ruins of the gas molecule belonging to the electronic nose technology is identified with sensors designed and chemical processes in the cell within the mini device. It's not the reaction that occurs with a wired or wireless connection. The device that communicates with a mobile device converted data can be read through the software. Cell phones, tablet devices and the extra software to be uploaded can make the data taken. Suspected remains of vegetable or fruit from the supermarkets or from home will be put as a tiny piece on analysis tank and then the sensors are substituted by a series of perceived assets processed and through the other device with pre-installed software can make appear the result. This device is in the testing phase yet but as in used finding pesticide active ingredients in agricultural production, only organophosphates and carbamates have been reported in the analysis of pesticides. The sensor is used for the organophosphates and carbamates determination of pesticides. Organophosphates and carbamates pesticides are not only chemicals there are also another chemical groups also used in drugs. These drugs are belongs to other pharmaceutical group other such as the chlorinated compounds, synthetic pyrethroid, Amine and hydrazine derivatives, organic Tin, sulfur compounds, triazoles are pesticides, etc.

Electronic nose systems can be used only if there is essential compounds in the environment. It does not include every pesticide has active ingredient molecule as essential gas molecule. In such a case, the non-pesticide active substances in the samples are undetectable and the device will give false results.

The examples of agricultural products used in the device gotten from the sales points. Ripped from source and/or waited product on the shelf or/and in the store for a while can cause ranges of serum concentration of SAP.

Depending on the product to the wait time has an in-house pesticide active ingredients can be measured within the biological destruction and decreasing the amount of active substance and structure changing, indicating that it is a very dangerous pesticide metabolite.

Pesticide active ingredient in agricultural production falls below a certain figure can cause them pretend as hormones in the human body, this can cause serious health problems which is mentioned in variety of medical research. In addition, the pesticide metabolites which can be harmful to human health cannot be analyzed according to the introduction of the device.

This device can only work with the samples on sale point products and cannot be used for finding the pesticide or/and the residue of active matter metabolites from pesticide on agricultural products in the pre-harvest area

Electronic nose sensors on the device are easily affected by land conditions, ambient temperature and humidity because of that the calibration of the device gone bad. This device can be used for only at home for limited pesticide varieties and with small amount of sample, it is not suitable for using it for big tonnage for various of pesticide analysis. There is no display and operating system device itself. Process data can be read of with the second needs as a mobile device. The software for the analysis is not in the main device we will need a second device for it.

In Turkish Patent Institute with the number 2010-05568 recorded in the patent application documents of reference mentioned before the harvest will make Multiple Pesticide Residue analysis of plant Hand-held measurement device. We didn't continue processing that patent because we will be technically insufficient and exceeding the desired specifications and prompts by us.

The mentioned device hasnot got recording program for the land and the GPS system which will detect the land the coordinates. A separate registration system needed for data entry, and there should be another computer. Device communication with different terrain conditions in the main central computer and internet connection which will be allowed with a sim card to the card slot and motherboard hadn't been in the system which is required. The results of the analysis to be done within the device as there is no mini printer that will allow writing; You must be connected to a separate printer device as needed. On the screen and the menu control keypads are located separately, without the keypad on the touch screen menu for single use is not possible and also not possible to use vertical, horizontal screen when needed.

Durable and lightweight material mentioned in the outer surface of the device, cannot to protect from external factors such as corresponds to water, dust and moisture. For this reason, open field conditions, such as in wet weather, intensive moisture in greenhouse environment use the summer term, the prevailing wind is blowing open to agricultural land can be experienced difficulties in protecting against dust in the camera's penetrating and unnoticed, dust particles, water droplets and moisture on the inside of the device, malfunction of electronic components located on metal particle with time cause oxidation device, depending on the moisture in the compromise, the analysis will lead to incorrect results in.

In the land of the aforementioned device is not suitable for dust, water and moisture during harvest, before the land and location information of agricultural products over the same device analysis recorded and live plant tissue the pesticide active ingredient pesticide and/or metabolites analysis.

The company named SEACOST SCIENCE. Inc—A CHEMICAL SENSOR COMPANY developed MEMS based micro electro mechanical systems analysis in organic compounds, toxic industrial chemicals, nitro compounds, and carbon dioxide. The device which is mentioned designed as a sensor of electronic nose which can identify variety of ingredients including essential compounds at the molecular level in its mini gas chamber.

The sensors on the device can only analyzed gas forms and organophosphate and carbamates compounds. Organophosphate and carbamates in agricultural production not only pesticide also used drugs other chemical groups. In the agricultural pesticide usage cannot be limited only organophosphates and carbamates drugs but also chlorinated compounds, synthetic pyrethroid, Amine and hydrazine derivatives, organic Tin, sulfur compounds, triazoles are pesticides, etc.

This product is also unable detect metabolites with the electronic nose sensor which are very dangerous for human health and can be seem only the reaction of pesticide with SAP of the plant.

The mentioned device can only work in % 0-60% relative humidity range and 25C-35 C range, with relevant results made under these conditions. During agricultural products are harvested climatic conditions may not fit the device working conditions all the time. As an example, in the greenhouse during the summer period the temperature during harvest time at least 45C and humidity is 70%.

Open field may not be able to provide these conditions at harvest time as well. In the morning heat changes will be 0C to 50 C and/or intense rainfall can cause 100% relative humidity during harvest land. I will effect the electronic nose sensor sensitivity of this device the results of the analysis will not be healthy.

In this device there can only be detected carbamates pesticide Carbaryl. Therefore, with this device, we are unable to detect metabolites and other harmful pesticides which not contains gas molecule.

Electronic nose systems can be used if only the environment has essential gas forms.

It does not include every pesticide has got essential gas molecule. In such cases, pesticide cannot be detect and the device will give false results.

Electronic nose sensor on the device is different from the electrochemical analysis methods of voltammetric analysis methods.

The device reported as using 900 MHZ wireless connection. 900 MHZ is not a good choice for connection of land, it is more efficient indoors. With the increasing population, this technology is no longer a perfect choice of technological life.

The topography of different terrains and agricultural production are mostly outside of downtown. You may not have the necessary infrastructure to transmit data for each production area.

For a quality data process, usage and transmit in open field measurements, we need at least 1800 MHZ connection speed, otherwise there may some problems occur and if there is inability to have 1800 MHZ connection speed, the device should contact using satellite to deliver the data to the main center.

This device does not have software to show different pesticide groups and active pesticide metabolites at the same time and also device can only work in 0%-60 moisture and 25C-35 C degrees' air temperature.

This device also cannot record sample information, where it from, who does it belong to, analysis date and hour. It needs another computer record system. The outcomes from this device, (unless is there another record system) doesn't have those information, therefore we are unable to have momentarily informed about the situation and we are also unable to make anything with the negative results to the responsible.

The reasons mentioned above, make this device unuseful for our purpose, such as making multible pesticide analysis and pesticide metabolite analysis on the field.

In the market currently to be sold hand-held pesticide analysis devices. This device can detect changing colors in their kit and then covert it numeric data based on organophosphates and carbamates enzyme activity, it may take 10 minutes.

In the agriculturel pesticide usage cannot be limited only organophosphates and carbamates drugs but also chlorinated compounds, synthetic pyrethroid, Amine and hydrazine derivatives, organic Tin, sulfur compounds, triazoles are pesticides, etc. These active matters cannot be detect with enzyme reaction method, needed to be used different methods to detetct them.

Diagnosis value starts at least 100 level upper than legal MRL limits and the enzymes which are in the device needs the optimum conditions.

Changing climate and geographic conditions can cause kit is broken for enzyme activity to give healthy results. You cannot record any information about the sample on this device, there is needed a secondary record system for it. Data transfer for device also can be made only with secondary computers.

These devices are not usefull for all kinds of terrain, climate and geographical for analysing pesticides and/or pesticide metabolites.

TAIWO SCIENCE, Inc. by USDA-National Institute of food and agriculture in the United States (National Institute of Food and Agriculture) takes advantage of the grants funds supported by An Inexpensive Mems-Based Pesticide Detector For Long Term Monitoring and titled 2006-United States Department of Agriculture USDA's reference number-00188 project web page, its subsidiaries, is The Research, Education and Economics Information System (REEIS) and The Current Research Information System (CRIS) page www.reeis.usda.gov/crisprojectpages 0206567 with the number accepted. This project is selected in 3 target detection of electronic nose-enabled sensors effects of pesticides for work. Dichloropropene, methyl bromide and selected pesticides methyl isocyanate. All three are applied to all creatures in the area featured in the formulation of essential gas fumigant that kills compounds. Is applied commercially in the form of a tablet or tube into effect by passing gas in the environment. Human and all other living creatures due to the dangerous storage conditions and subject to the conditions are heavy pesticide application principles. During application it is not attention and conscious application isolation is not done with the acute effects can lead to mass deaths. In the long term, human, nature and all living creatures can lead to chronic problems, air, soil and water may cause poisoning.

In the mentioned project, it has been aimed for those 3 pesticides to be detected in the air by using sensors or developing of analysis methods and devices.

Pesticides are applied to agricultural land near the settlements and schools were asked to be created inspired air related security criteria. Continuous measurement of active gaseous pesticides in air aimed to improve farmer's awareness on usage of them.

The study is featured on electronic nose developed sensors that can detect volatile compounds. Using sensors and sensor has been developed on the principle of operation of the devices. All of the pesticides used in agricultural production does not contain volatile gas compounds and not in the air released directly from the plant is within: analysis methods are also different.

This project studies detecttion the presence of pesticides in the form of dangerous gas, but this devices and sensors cannot use for detection the pesticide in live plant tissue and/or it is not suitable to detect pesticide metabolites. It is also cannot be used for during harvest time.

Measurement, 2008, vol. 41, p. 44-54 published article mentiones hand-held spectrometric measurement applications for spectrometer. Spectrometers are devices which helps measuring the color change of the samples absorb substances for analysis. For accuracy of analysis, each sample has only one analysis. The mentioned spectrometer is not used for multiple pesticide active substances and active metabolites.

Analytica Chimica Acta, 2007, vol. 596, p. 1-8 published articles mentioned pesticides can be seen with optical methods for studies. The effect of plant physiological spectral matrix in order to overcome, in the form of nano and micro Crystal fluorescent labeling agent are granted and this crystal as the interaction of pesticides with spectral examined, within the existing pesticide plant of Fourier transform spectral method of more specific and accurate display. The mentioned device which is according to the spectral wavelength of pesticides technical in the light color detection is a method of analysis based on the aforementioned methods are pesticide analysis and electrochemical analysis methods is not only to ensure the accuracy of the detection of an active ingredient is mainly because more than one so multiple pesticide active ingredient in the analysis of the active substance and metabolites of electrochemical is unavailable.

### SUMMARY OF THE INVENTION

Finding harmful amount of pesticides and/or remains of pesticide active ingredient to human health in the offered agricultural products to the market can cause time consume, high cost, random selection of final products when it is already left the production area make it impossible to fix, difficult to exterminate,difficult to return the products,using unsufficent labor power, the rumors of not being exterminate and sold in local markets, commercial and economic loses, the legal sanctions. Our aim is making pesticides and/or remains of pesticide active ingredient analysis in harvest point with an electronic handheld portable Analyzer while the live instance from given land information of the product of the plant, the identity of the owner of the land, the history and analysis of the information of the person who made the analysis at the same time, register and get your own GPS system coordinates of the device the device is developed for conducting on-the-spot analysis with electrochemical analysis methods, the pesticide active substances and/or live plant active substances determination of metabolites and detection, the device can be read from the screen, together with the producers and land information printed output, with its own operating system of the device and sim card when necessary, without the need to transfer data to another computer, sharing of information, is intended to provide backup and storage.

### BRIEF DESCRIPTION OF THE DRAWINGS

FIG. 1 is a front view of one preferred embodiment of the portable electronic pesticide residue analysis device.

FIG. 2 is a rear view of one preferred embodiment of the portable electronic pesticide residue analysis device.

FIG. 3A is a view of one preferred embodiment of a first interior portion of the portable electronic pesticide residue analysis device.

FIG. 3B is a view of one preferred embodiment of a second interior portion of the portable electronic pesticide residue analysis device.

FIG. 4 is a side view of one preferred embodiment of the portable electronic pesticide residue analysis device.

FIG. 5 is a top view of one preferred embodiment of the portable electronic pesticide residue analysis device.

FIG. 6 is a bottom view of one preferred embodiment of the portable electronic pesticide residue analysis device.

### DETAILED DESCRIPTION OF THE INVENTION

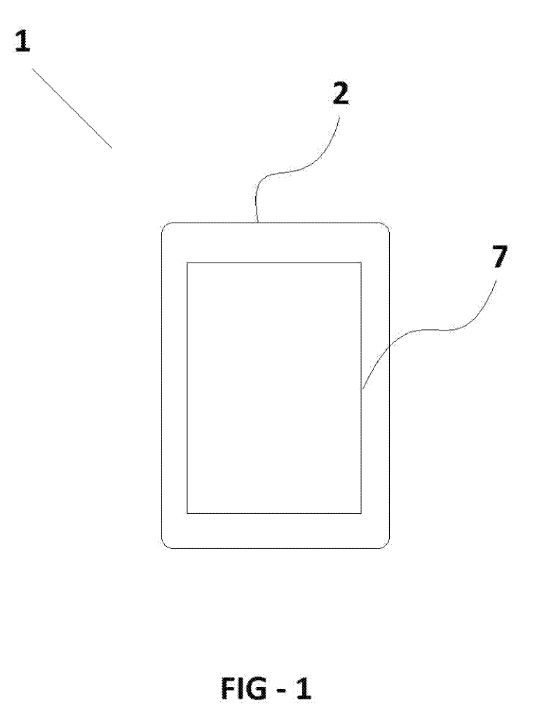

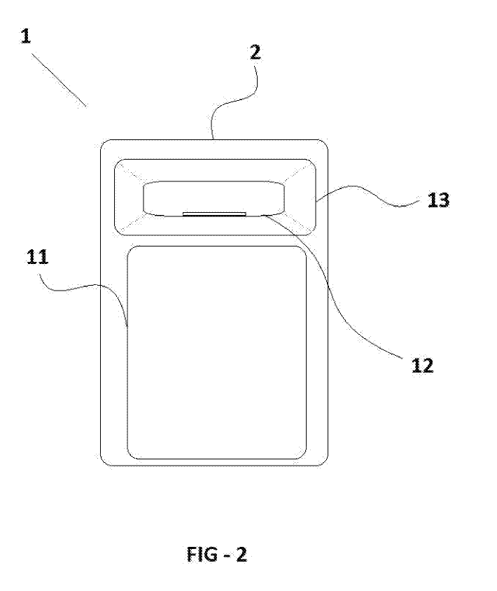

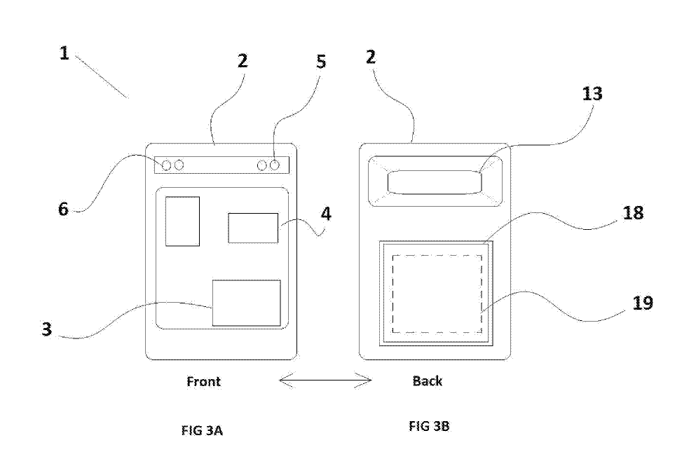

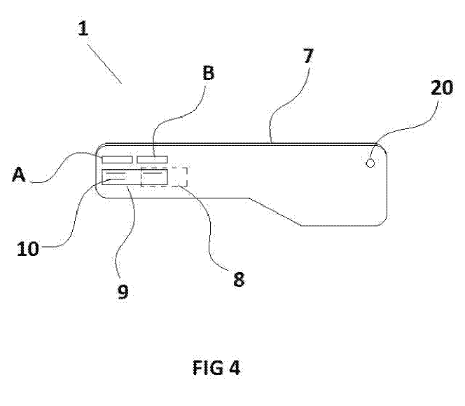

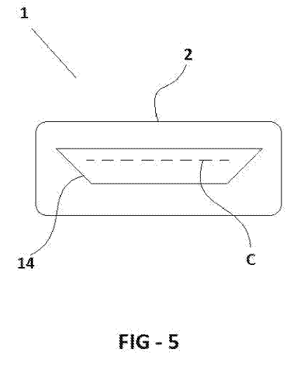

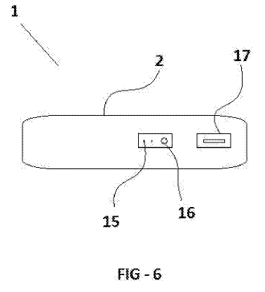

As shown in FIG. 1, FIG. 2, FIG. 3 and FIG. 4; a portable elecktronic pesticide residue analysis device (1) which will determine the pesticide or/and active pesticide ingiridient metabolite with method of electro-chemical on the live plant at the field or at the land before harvest is made of durable and lightweight material, water, dust, and moisture-proof, short term water drops, splashes, resistant to water pressure low and high intensity short term external body part (2); the system will run inland and will perform the analysis process, the resulting data can be read on digital devices will convert the values to the device software enhancement MRI SCANS installed graphene and/or super fast high carriers containing compound transition metals-based micro-processor, graphics processor, microcontroller, memory and electronic circuit chips, hard drive on which the operating system is installed, the screen (7) of the GPS microchip card, micro sim card slot (10) on the printer processor, a motherboard and/or card from the group; bumps, drops and so on cases analysis done on the device in order to take the fall to protect the process of multiplication by detecting negative situations, etc about the procedure automatically until the device will freeze and normal use deletion to protect against sudden motion sensor (5) puts the protective system; according to the level of light in the environment that sets the screen (7) brightness of the device ambient light sensor to adjust; the use of the programs for the operating system of the device and allows access to, water, dust, and moisture-proof, short term water drops, splashes, resistant to low and high intensity short term water pressure, fingerprint stain repellent, the entire process, information introduction was written by tapping the on-screen keyboard of the finger and the finger movements of the menu dial, can be made both numerical and graphical image operations, vertical and horizontal use capacitive touch screen (7); will multiple pesticide analysis device (1) off when not in use and water, protection against dust and humidity, and automatically locked mini sliding lid (8) preserved one or more of the test kit is to be used for regular calibration calibration the device of the kit to be worn while working and possible external factors that enable protection from water, dust, and moisture repellent nano-material, covered with full contact on the tip and entry kit slot sitting on each other is locked and sealed when covered with protective nano material provided the featured input socket (9); on the main body (2) to dust, water and moisture resistant standard sim card (A) and memory card is installed, press a couple of card holder of the extraction system; it's in the back of the protective device, water, dust and moisture-resistant protective cover made of durable and lightweight material that featured; when the paper roll is located under the hood that allows a change of a mini printer roll holder; when not in use, protection against water, dust and humidity property on the output of the printer paper closes snugly with seat cover; when you need additional data to the device in order to provide the entry of water when not in use, protection against dust and humidity sitting snugly on the property when installed under the hood to provide a complete sealing mini protector-Bay (15) data cable entrance; have the same protective cover the protective property RS485 and/or different port entrance; the battery (19) is located under the protective cover of the back of the device pane; it is located under the protective cover of the back of the device 5 field battery (19) pane; there is rechargeable smart battery (19) which can provide 8 hours power and at least 1000 analysis, it can stand on stanby mode for 24 hours; located on the side of the device and a device to turn on/off button (20).

You can find the analyzing method of the portable elecktronic pesticide residue analysis device (1) which will determine the pesticide or/and active pesticide ingiridient metabolite with method of electro-chemical on the live plant at the field or at the land before harvest below;
opening of the device with the on/off button (20),
continue to, the device screen (7) by entering the user code and password onto the page that opens,
continue to, with the adoption of the code and password opening analysis home page,
to begin the process, analysis on the main window is registered in the database of the Ministry of the manufacturer ID and/or the manufacturer's registration number must be written,
continue to, on the page that opens the gps system is activated by the system brought to harvest and saving of measurement will be made of land information,
continue to, selecting the product to do the analysis menu analysis,
continue to, placing the device installed on the device for the analysis kit system software by opening the slide auto kit holder,
continue to, the analysis set should be placed in the slot of the tip,
with the introduction of the software installed on the device the process analysis kit instance located at the selected products occurs on the pesticide we brought the reaction of active kit electric signals according to the pesticide active ingredient will be chosen automatically installed on the device the analysis program into electrochemical analysis methods of data,
continue to, the pesticide active ingredient contained in the analysis kit metabolites detected in electrochemical sensors of detection of installed software on the device according to the metabolite will be activated automatically by analyzing data from into,
continue to, at the end of the time set, the data obtained through the software on the device can be converted to numeric values,
the numeric value of the process which the analysis data detected with active ingredient turned into a chart table with values of the device memory to be saved,
continue to, the analysis report can be registered with information of land, manufacturer and product info to the public body or/and related firm's database with the software which is in the device and wireless connection.
continue to, with mini printer installed on the device to print on the election of the results of the analysis, the results are printed all the analysis and/or risky of them,
continue to concluding the analyzing for that sample which is shown on the analyzing page.
continue to, in the analysis page on the analysis menu guide and removing the analysis kit from the device and with cover will be automaticly closed,
for starting the program, it needs the current membership of the user code and password, registering the info of product, manufacturer and land
during the process, as required by the installed software on the device program feature analysis once the user process any data in the case of faulty operation, not being able to delete, cancel the error during device screen (7) warning and error correction can be continuing with; this process can also be saved to system memory,
with the installed protective software of analysis device (1), during the hit or such cases with the sensor detects the screen (7) blurring and executed the procedure as freeze protection process; normal conditions of use, it is advisable to continue where it left off when it comes to processing,
at the level of 10% of the battery (19) power level of device, in which case it has audible and light warning,
the battery (19) power level is 2% of cases, audible and light warning given as critical power and there can be made only one more analysis in the analysis device (1), the previous and the current data taken for protection.

The preferred application of device use for detecting pesticide and/or active pesticide ingredient metabolites, with the taken SAP, stick, trunk, leaves, shell, fruit pieces just before the harvest from plant and with the help of mechanisms based on the analysis kit will be dismembered them and put in a series of process and with the electro sensore of device reacts and convert it numeric data which can be read.

As a method of analysis on the device to be identified according to the pesticide active ingredient, a variable of the desired electro-chemical analysis techniques will be used. Electro-chemical method of electrode-solution system response of the system is measured by performing the electrical effect.

These methods are accepted with the scientific researchs in the most economical and appropriate analysis for pesticide analysis methods.

In the electrochemical analysis methods, voltammetryc methods are the preferred technique,on behalf of the device's advantage; it is possible to work with very little samp, it is not always a need for a sample preparation process, some samples can be rolled directly into analysis, with the lower limit of determination makes the analysis much more sensitive, selectivity and accuracy is better, hand-held device for multiple pesticide analysis for non-liquid and liquid samples allow the development of new analysis methods, it can work with different electrode so electronic portable hand-held device is intended for use in the analysis of multiple pesticides.

In the Voltammetryc method, especially in Dithiocarbamate pesticides and appointed methods, there are used the electronic nose sensor which is detecting ETU metabolites with gas molecules method. Besides, with these method, it is possible to analysis of pesticide residue during the analysis or at the same time. In these points it is different from the enzyme base biological there or not there analyzing method.

Voltammetry is an indicator or work under the circumstances applied polarized electrode potential as a function of flow measurement from the analysis sample from a group obtained information about electro analytical method.

Voltammetric experiments is performed at trio electrode system. One of the three electrode will be potentially modified to indicator electrode in time or working electrode.

It is used as an indicator electrot in voltammetryc are mercury, Platinum, gold, Palladium, carbon electrode (electrode, glassy carbon graphite, carbon cake). The second electrode in electrode system is the potential electrode which remains constant during the experiment.

The reference electrode is usually, Ag/AgCl or saturated calomel electrot. The third electrode in the electrode system is contrast or auxiliary electrode. As the contrast electrode it is usually used P1 wire.

In Voltammetric analysis method, the working electrode occurs as a result of the reduction of the items or increased. The stream which is resulted of decreasing called as cathodic stream and the stream which is resulted of increasing called as Anodic stream.

In the device as an electrode system of analysis is prepared for the requested pesticides analysis. There will be used electrode and electronic circuit components which is made of graphene which is shorten the analysis time and increasing the speed of the action, nanopolymer and/or transition metal compound high carrier weight Super fast electrode. Analysis set sets within the analysis work in electrochemical sensor reacting with samples, reference and comparison of electrodes on the surface of the sensor and/or located on either side of the device will connect to the party that the surface of the electrodes are thin cables. Device is being contacted and analysis Kit that part appear to be occurring in the reaction device sensor to connect a separate computer with the processor on the device without having to read electronic and computer software is software that is processed with capacitive touch screen (7) can be read as both numeric and graphic table, visible, printable, forwarded data.

These electrodes both surface area and working, reference and auxiliary electrodes are gold, silver, Platinum, carbon, and more are preparing to be the desired item. Standardization, being provided easly, laminated with nano materials, the results are more accurate and sensitive makes these electrodes more desirable in voltammetryc analysis.

In the market, we can find various electrodes in various models from various companies and they are incaple of being fold and bend. So we aimed to develop these electrodes for using suitably in the device which is used in field and also they are easy to carry and easy to store.

The main voltametrik methods which are going to be used in the device;
1. Linear Scanning Voltammetry LSV
2. Differential Pulse Voltammetry DPV
3. Square-Wave Voltammetry SWV
4. Normal Pulse Voltammetry NPV
5. AC Voltammetry ACV
6. Cyclic Voltammetry CV
7. Anodic Stripping Voltammetry ASV
8. Adsorptif Stripping Voltammetry AdSV
9. Adsorptif Catalytic Stripping Voltammetry AdCSV
10. Chronopotentiometric Stripping SCP

Voltammetric analysis of the device, the measuring bar electrode technology, new production techniques and developments in nanotechnology is held in front of the eyes of amperometryc intended analysis.

The device is also used as a measuring stick on the set of the analysis Kit electrochemical electrode sensor will be developed with new kit systems and facilitating the use of the software program moving mercury and mercury electrode type is based on the principle of polarographic study of the kit system analysis is intended to be done.

With the device's hand-held model makes the device can be always carried by the user and at any possible analysis is intended to be able to do this.

The device's dust, water and moisture-proof, short water falls and water-resistant material-coated lightweight and resistant to water pressure inside made from material that allowes the device for different climates and topography terrain conditions and also from external factors is intended to ensure the protection and ease of transport to the user.

On the inside of the device there are A/D, D/A converter, SPI, Graphene and/or super fast high carriers containing compound transition metals-based micro-and graphics-processor electronic circuits, RS485 and/or other ports, the operating system hard disk, the sudden motion sensors (5) set up ambient light sensors, processor processor, the video card, mini printer processor is the use of multiple card system can be shrunk to the size of the device, only one device can be executed simultaneously by more than one analysis, digital-to-analog conversion of digital-to-analog data of the data, graphics software program integrated spreadsheet operations can be run, micro processors, converter and capacitive touch sensors work in a synchronized format of the screen (7), the screen (7) is capacitive touch screen (7) of the software run, simplifying the analysis processes, a sudden drop in such cases, the impact of the device the device must be taken to protect the process, analysis, operation of the device made to normal working conditions until the freezing without loss of data, the analysis of the results can be printed from the device and, if necessary, the device can be sent to external printer with a wireless internet connection.

It is aimed to increase the sensivity of the analysis and speed of processing, there are used nanopolymer and/or super fast high is a compound that contains metals-based carriers in the electronic circuits and processors.

With the sudden motion sensors (5) on the device, in such cases as a sudden drop and an impact, the sensor-driven protector software program activated and freeze all the process steps, analysis and data until the normal conditions restored and it will continue the operation where it is left.

Set the ambient light sensors (6) on the device with automatic adjustment of display brightness according to ambient light and the battery (19) life saver is intended to provide.

The the GPS processor in the device made link with the satellite on the land is intended to be saved to the device coordinate plane by utilizing.

The device is the main card printer program within the processor, use a mini printer and the analysis results with the required data on the device using the specially developed printer program without having to a separate printer connection, results are intended to be printed.

The device is a mini printer roll in the slot, the analysis of the results across devices as needed using the program typed the printer processor document can be retrieved in order to hook up the printer output (C) is intended for mini rolls.

The device is different from the devices which are already on the market in the case of they need another software which can be used in computer, the device has own software and screen (7) which let you to see the results.

Android on the device, Windows, IOS etc. through the use of the operating program, different geographical conditions and protected production areas in open terrain data, sharing information, program updates, location positioning and operating-system update for downloads without a separate device connected to the computer through touch-capacitive screen (7) related programs can be loaded and can be done using the device menu of operations.

Device with sim card slot (10) in the different geographic conditions for wireless data transfer as needed, some of the nearby base station will provide satellite connections, internet connection and/or sim card (A) can be used to hang out and operating programs.

In the device memory card slot (10) that belongs to the process data is stored on the device can be enlarged capacity of the memory card can be inserted and the device in order to be used.

The capacitive touch screen (7) the user has a separate keypad and key keyboard without the need to capacitive screens (7) with multi-touch feature using your fingers across the screen (7) can be selected and installed before the system menu in order to make the system reach the data entry to the program, transactions can follow through on the screen (7) again, the use of the page on the screen (7) device horizontally and vertically in order to select the relevant menu and can use the operating system installed on the device.

The kit is the input slot (9) on the end, will make multiple pesticide analysis analysis set set/sets and routine calibration operations for attaching analysis and calibration calibration kit is intended to be made.

The data transfer cable (USB) and R485 and/or other person using the device of the entrance to the port and the business centre of institutions when necessary, upon receipt of the data obtained and to share, in cases where the software and the device are required outside of a wireless internet connection to the aim and a separate computer can install new programs, the additional hardware device and when the need to be able to connect to the devices, the need arises to projectors by connecting directly with the information reflected on the device can be used in presentations and both approaches with the same component on the device, and to achieve savings.

Other ports are RS485 device and/or through the use of the device will work on field conditions and grounding problems voltage fluctuations in the RS232 interface, signal to noise caused by the prevention of the problem of security analysis, communicate with each other with the card protocol converter RS485 device providing the connection between the unit and the sensor and scientific developments can be used of the cards other than depending on the RS485.

The device's printer roll, kit entry, sim card (A) and memory card (B), USB and port entrances self sliding caps through the use of the device ensuring the preservation and analysis accuracy protection from external factors.

With the battery (19) of the device pane, will provide power to the device can be attached to the device of various power battery (19).

Run the device for at least 8 hours provide the power to the device is opened if the device holds a 24 hour stand by ready for analysis, the test can be done about 1000 times, rechargeable and use the smart battery (19) specifications battery (19), the device field conditions proper to study long hours and needed to be ready at any moment measuring aimed to provide.

The on/off button (20) when prompted by pressing the button the device by opening the analysis needs to be done in 10 seconds and finished within 10 seconds by pressing the button completely the closure is intended to provide.

The analyzing device which is mentioned to serve the qualifications above can be produced by any field of industry which will also be useful and it can be also applicable for industrial purposes.

### REFERENCED PARTS

1. Pesticide analysis device
2. Body part
3. Hard disc
4. Cards
5. Motion Sensor
6. Light sensor
7. Screen
8. Sliding lid
9. Input Slot
10. Card slot
11. Protective lid
12. Roll lid
13. Roll slot
14. Valve
15. Mini protector-bay
16. Cable entry
17. Port entry
18. Battery compartment
19. Battery
20. On/off button
   A. Sim card
   B. Memory card
   C. Out of the printer

1. Before the harvest in agricultural production in the field and live plant will be chosen in the electro-chemical methods pesticide active ingredient pesticide metabolites residue analysis and/or do portable electronic pesticide residue analysis is the analysis method to be used in property:
opening of the device with the on/off button,
in the process, the device screen by entering the user code and password onto the page that opens,
in the process, with the adoption of the code and password opening analysis home page,
to begin the process, analysis on the main window is registered in the database of the Ministry of the manufacturer ID and/or the manufacturer's registration number must be written,
in the process, on the page that opens the GPS system is activated by the system brought to harvest and saving of measurement will be made of land information,
in the process, selecting the product to do the analysis menu analysis,
in the process, placing the device installed on the device for the analysis kit system software by opening the slide auto kit holder,
in the process, the analysis set should be placed in the slot of the tip,
with the introduction of the software installed on the device the process analysis kit instance located at the selected products occurs on the pesticide we brought the reaction of active kit electric signals according to the pesticide active ingredient will be chosen automatically installed on the device the analysis program into electrochemical analysis methods of data,
in the process, the pesticide active ingredient contained in the analysis kit metabolites detected in electrochemical sensors of detection of installed software on the device according to the metabolite will be activated automatically by analyzing data from into,
in the process, at the end of the time set, the data obtained through the software on the device can be read, processed and converted to numeric values,
the numeric value of the process, analysis, data detected with active ingredient turned into a chart table with values of the device memory to be saved,
in the process, the analysis of installed software on the device state provider program and wireless internet connections and related land, the company's database product manufacturer, along with the site by registering,
in the process, with mini printer installed on the device to print on the election of the results of the analysis, the results are printed all the analysis and/or risk,
more on the device screen analysis process page for an example of the analysis process of concluding that,
in the process, the process analysis on the analysis menu routing and analysis kit from the device with the cover of automatic closure kit,
the current membership of the user code and password to begin processing, product manufacturer, and the system of land and although this pretreatment steps then analysis program can be started,
during the process, as required by the installed software on the device program feature analysis once the user process any data deletion, cancellation, in case of faulty operation during the device screen warning and error correction can be continue with; This process can also be saved to system memory,
the reduction of the device during device installed protective software analysis and so on cases with the introduction of the sensor that detects the screen blurring and executed the procedure as freeze protection process; normal conditions of use, it is advisable to continue where it left off when it comes to processing,
at the level of 10% of the battery power level device, in which case it is audible and light warning,
the battery power level is 2% of cases, audible and light warning and critical power levels can be made only at the device analysis, taking the previous and current data protection process steps.

2. Before the harvest in agricultural production in the field and live plant will be chosen in electrochemical methods remains of pesticide active ingredient pesticide and/or metabolites analysis is pesticide residue analysis of portable electronic device will feature: durable and lightweight material, water, dust, and moisture-proof, short term water drops, splashes, resistant to water pressure low and high intensity short term external body part; the system will run inland and will perform the analysis process, the resulting data can be read on digital devices will convert the values to the device software enhancement MRI SCANS installed Graphene and/or super fast high carriers containing compound transition metals-based micro-processor, graphics processor, microcontroller, memory and electronic circuit chips, hard disk on which the operating system is installed, a card is the microchip, GPS, micro sim card slot on the printer processor, a motherboard and/or cards group; bumps, drops and so on cases analysis done on the device in order to take the fall to protect the process of multiplication by detecting negative situations, etc about the procedure automatically until the device will freeze and normal use deletion to protect against sudden motion sensor motion sensor puts the protective system; sets the screen brightness according to ambient light ambient light sensors; the use of the programs for the operating system of the device and allows access to, water, dust, and moisture-proof, short term water drops, splashes, resistant to low and high intensity short term water pressure, fingerprint stain repellent, the entire process, information introduction was written by tapping the on-screen keyboard of the finger and the finger movements of the menu dial, both numerical and graphical images can be made suitable for the vertical and horizontal operation of capacitive touch screen; will multiple pesticide analysis device off when not in use and water, protection against dust and humidity, and automatically locked mini sliding lid preserved one or more of the test kit is to be used for regular calibration calibration the device of the kit to be worn while working and possible external factors that enable protection from water, dust, and moisture repellent nano-material, covered with full contact on the tip and entry kit slot sitting on each other is locked and sealed when covered with protective nano material provided the featured input slot; on the main body to dust, water and moisture resistant standard sim card and memory card is inserted, discharge of the sticker system a dual card slot; It's in the back of the protective device, water, dust and moisture-resistant protective cover made of durable and lightweight material that featured; when the paper roll is located under the hood that allows a change of a mini printer roll slots; when not in use, protection against water, dust and humidity property on the output of the printer paper closes snugly seated valve; when you need additional data to the device in order to provide input when not in use, protection against water, dust and humidity sitting snugly on the property when installed under the hood to provide a complete sealing mini protector-Bay data cable entry;
have the same protective cover the protective property RS485 and/or different port entry;
located at the bottom of the back cover of the device's various protective power battery compartment; at least 1000 enables at least 8 hours continuous measurement device capable of running and active stand by position, they can expect at least 24 hours have rechargeable and smart battery characteristics battery; located on the side of the device and a device to turn on/off button.

3. According to claim 2, before the harvest in agricultural production, in the field and live plant will be chosen in the electro-chemical methods pesticide active ingredient pesticide metabolites residue analysis and/or portable electronic pesticide residue analysis is the device that will make property: durable and lightweight material, water, dust, and moisture-proof, short term water drops, splashes, resistant to water pressure low and high intensity short term external body part.

4. According to claim 3, before the harvest in agricultural production, in the field and live plant will be chosen in the electro-chemical methods pesticide active ingredient pesticide metabolites residue analysis and/or portable electronic pesticide residue analysis is the device that will make property: the system will run inland and will perform the analysis process, the resulting data can be read on digital devices will convert the values to the device software enhancement MRI SCANS installed Graphene and/or super fast high carriers containing compound transition metals-based micro-processor, graphics processor, microcontroller, memory and electronic circuits, chips, the hard disk on which the operating system is installed, the GPS microchip, the video card, the processor micro-printer, movement and ambient light adjustment sensor processors, the motion sensors comes in now will get software to protect data in the device, set the ambient light sensor is now sticking of environment will set the screen brightness according to software brightness and a sim card slot on the mainboard.

5. According to claim 4, before the harvest in agricultural production, in the field and live plant will be chosen in the electro-chemical methods pesticide active ingredient pesticide metabolites residue analysis and/or portable electronic pesticide residue analysis is the device that will make property: setting up the GPS device, movement and ambient light sensors, circuits, depending on the motherboard of the printer circuit and processing electronic circuit boards.

6. According to claim 5, before the harvest in agricultural production, in the field and live plant will be chosen in the electro-chemical methods pesticide active ingredient pesticide metabolites residue analysis and/or portable electronic pesticide residue analysis is the device that will make property: bumps, drops and so on cases analysis done on the device in order to take the fall to protect the process of multiplication by detecting negative situations, etc about the procedure automatically until the device will freeze and normal use deletion to protect against sudden motion sensor that puts protective system motion sensors.

7. According to claim 6, before the harvest in agricultural production, in the field and live plant will be chosen in the electro-chemical methods pesticide active ingredient pesticide metabolites residue analysis and/or portable electronic pesticide residue analysis is the device that will make property: detects the device display environment lightness brightness automatically adjusts the ambient light detection sensors.

8. According to claim 7, before the harvest in agricultural production, in the field and live plant will be chosen in the electro-chemical methods pesticide active ingredient pesticide metabolites residue analysis and/or portable electronic pesticide residue analysis is the device that will make property: the use of the programs for the operating system of the device and allows access to, water, dust, and moisture-proof, short term water drops, splashes, resistant to low and high intensity short term water pressure, fingerprint stain repellent, the entire process, information introduction was written by tapping the on-screen keyboard of the finger and the finger movements of the menu dial, both numerical and graphical images can be made suitable for the vertical and horizontal operation of capacitive touch screen.

9. According to claim 8, before the harvest in agricultural production, in the field and live plant will be chosen in the electro-chemical methods pesticide active ingredient pesticide metabolites residue analysis and/or portable electronic pesticide residue analysis is the device that will make property: will multiple pesticide analysis device off when not in use and water, protection against dust and humidity, and automatically locked mini sliding lid preserved one or more of the test kit is to be used for regular calibration calibration the device of the kit to be worn while working and possible external factors that enable protection from water, dust, and moisture repellent nano-material, covered with full contact on the tip and entry kit slot sitting on each other is locked and sealed when covered with protective nano material provided the featured input slot.

10. According to claim 9, before the harvest in agricultural production, in the field and live plant will be chosen in the electro-chemical methods pesticide active ingredient pesticide metabolites residue analysis and/or portable electronic pesticide residue analysis is the device that will make property: on the main body to dust, water and moisture resistant standard sim card and memory card is inserted, discharge of the sticker system a dual card slot.

11. According to claim 10, before the harvest in agricultural production, in the field and live plant will be chosen in the electro-chemical methods pesticide active ingredient pesticide metabolites residue analysis and/or portable electronic pesticide residue analysis is the device that will make property: it's in the back of the protective device, water, dust and moisture-resistant protective cover made of durable and lightweight material featured on the.

12. According to claim 11, before the harvest in agricultural production, in the field and live plant will be chosen in the electro-chemical methods pesticide active ingredient pesticide metabolites residue analysis and/or portable electronic pesticide residue analysis is the device that will make property: when the paper roll is located under the hood that allows a change of a mini printer roll slot.

13. According to claim 12, before the harvest in agricultural production, in the field and live plant will be chosen in the electro-chemical methods pesticide active ingredient pesticide metabolites residue analysis and/or portable electronic pesticide residue analysis is the device that will make property: when not in use, protection against water, dust and humidity property on the output of the printer paper closes snugly with seat cover.

14. According to claim 13, before the harvest in agricultural production, in the field and live plant will be chosen in the electro-chemical methods pesticide active ingredient pesticide metabolites residue analysis and/or portable electronic pesticide residue analysis is the device that will make property: when you need additional data to the device in order to provide input when not in use, protection against water, dust and humidity sitting snugly on the property when installed under the hood to provide a complete sealing mini protector-Bay data cable entry.

15. According to claim 14, before the harvest in agricultural production, in the field and live plant will be chosen in the electro-chemical methods pesticide active ingredient pesticide metabolites residue analysis and/or portable electronic pesticide residue analysis is the device that will make property: have the same protective cover the protective property RS485 and/or different port entry.

16. According to claim 15, before the harvest in agricultural production, in the field and live plant will be chosen in the electro-chemical methods pesticide active ingredient pesticide metabolites residue analysis and/or portable electronic pesticide residue analysis is the device that will make property: sundry power battery compartment.

17. According to claim 16, before the harvest in agricultural production, in the field and live plant will be chosen in the electro-chemical methods pesticide active ingredient pesticide metabolites residue analysis and/or portable electronic pesticide residue analysis is the device that will make property: device for a period of at least 8 hours provide the power to run the device is opened if the device holds a 24 hour stand by ready for analysis, the test can do about 1000 life, rechargeable and smart battery features battery.

18. According to claim 17, before the harvest in agricultural production, in the field and live plant will be chosen in the electro-chemical methods pesticide active ingredient pesticide metabolites residue analysis and/or portable electronic pesticide residue analysis is the device that will make property: located on the side of the device and a device to turn on/off button.
